SEQUENCE LISTING (10 sequences)

SEQ ID NO: 1 - protein, 119 aa, mouse
EVKLVESGGGLVKPGGSLKLSCAASGFTFSTYAMSWVRQTPEKRLEWVASISTGDNTYYTDSVRGRFTISRNNARNILYLQMSSLRSQNTAMYFCTRGRGNRGDLFGYWGQQTLVTVSS

SEQ ID NO: 2 - protein, 107 aa, mouse
DIVMSQSPSSLAVSVGKVTMSCKSSQSLLYRNQMNYLAWYQQKPGQPKLLIYWASTRESGVDRFTGSGSGTEFTLTSSVKAEDLTVYYCQQYTYPRTFGGGTKLEIR

SEQ ID NO: 3 - protein, 110 aa, mouse
QVQLKQSGPGLVRPSQLSITCTVSGFPLGVFVHWVRQSPGKGLEWLVIWKGGNTDYNAAFMRLRISKDNSKSQVFFMNSLQTDDTAIYYCAVNLADAMDYWGQGTSIVSS

SEQ ID NO: 4 - protein, 102 aa, mouse
DIQMTQSPASLSASVGTVTITCRSSGNIHGYAWFQQKQGKSPQLLVNTKALAEGVPSRFSGGSGTQFSLKINNLQPDFGIYYCQHHYDLPRFGGGTKLEIKR

SEQ ID NO: 5 - protein, 110 aa, mouse
QVQLKQSGPGLVRPSQLSITCTVSGFPLGIFVHWVRQSPGKGLEWLVIWKGGNTDYNAAFMRLRITKDNSKSQVFFMNSLQTDDTAIYYCAVNLADAMDYWGQGTSIVSS

SEQ ID NO: 6 - protein, 102 aa, mouse
DIQMTQSPASLSASVGTVTITCRASGNIHGYAWFQQKQGKSPQLLVNTKTLAEGVPSRFSGGSGTQFSLKINSLQPDFGNYYCQHHYDLPRFGGGTKLEIKR

SEQ ID NO: 7 - protein, 110 aa, mouse
QVQLKQSGPGLVRPSQLSITCTVSGFPLGTFVHWVRQSPGKGLEWLVIWRGGNTDYNAAFMRLRITKDNSKSQVFFMNSLQTDDTAIYYCAVNLADAMDYWGQGTSIVSS

SEQ ID NO: 8 - protein, 102 aa, mouse; identical to SEQ ID NO: 6

SEQ ID NO: 9 - protein, 11 aa, mouse
GRGDRGDLFGY

SEQ ID NO: 10 - protein, 9 aa, mouse
VNLADAMDY

(19) United States
(12) Patent Application Publication
(10) Pub. No.: US 2008/0299132 A1
(43) Pub. Date: Dec. 4, 2008

(54) Synergistic compositions for treating HIV-1

(75) Inventors: Changhua Ji, Cupertino, CA (US); Suryanarayana Sankuratri, San Jose, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 11/699,827

(22) Filed: Jan. 30, 2007

Related U.S. Application Data
(60) Provisional application No. 60/772,094, filed on Jan. 30, 2006.

(51) Int. Cl. A61K 39/395; A61P 31/18

(52) U.S. Cl. 424/172.1

(57) ABSTRACT

Synergistic pharmaceutical compositions for treating or preventing HIV-1 infections comprising anti-CCR5 monoclonal antibodies and CCR5 antagonists, viral fusion inhibitors or viral attachment inhibitors are disclosed. The compositions exhibit significant greater activity than is anticipated from the activity of either component alone. Also provided are methods for treating or preventing HIV-1 using the same.

### CROSS REFERENCE TO PRIOR APPLICATIONS

This application claims the benefit of priority to U.S. Ser. No. 60/772,094 filed Jan. 30, 2006 the contents of which are hereby incorporated in their entirety by reference.

### FIELD OF THE INVENTION

The present invention relates synergistic compositions comprising monoclonal antibodies which bind to the CCR5 receptor and low molecular weight allosteric antagonists which block viral entry into CCR5 expressing cells. The present invention further relates to methods for treating or preventing HIV-1 infection by co-administering monoclonal antibodies and low molecular weight allosteric antagonists of the CCR5 receptor

### BACKGROUND OF THE INVENTION

A-M. Vandamme et al. (Antiviral Chemistry & Chemotherapy, 1998 9:187-203) disclose current HAART clinical treatments of HIV-1 infections in man including at least triple drug combinations. Highly active anti-retroviral therapy (HAART) has traditionally consisted of combination therapy with nucleoside reverse transcriptase inhibitors (NRTI), non-nucleoside reverse transcriptase inhibitors (NNRTI) and protease inhibitors (PI). These compounds inhibit biochemical processes required for viral replication. In compliant drug-naive patients, HAART is effective in reducing mortality and progression of HIV-1 to AIDS. While HAART has dramatically altered the prognosis for HIV-1 infected persons, there remain many drawbacks to the current therapy including highly complex dosing regimes and side effects which can be very severe (A. Carr and D. A. Cooper, Lancet 2000 356(9239):1423-1430). Moreover, these multidrug therapies do not eliminate HIV-1 and long-term treatment usually results in multidrug resistance, thus limiting their utility in long term therapy. Development of new drug therapies to provide better HIV-1 treatment remains a priority.

The chemokines are a subset of the cytokine family of soluble immune mediators and are pro-inflammatory peptides that exert their pharmacological effect through G-protein-coupled receptors. The CCR5 receptor is one member of this family. The chemokines are leukocyte chemotactic proteins capable of attracting leukocytes to various tissues, which is an essential response to inflammation and infection. The name “chemokine”, is a contraction of “chemotactic cytokines”. Human chemokines include approximately 50 structurally homologous small proteins comprising 50-120 amino acids. (M. Baggiolini et al., Ann. Rev. Immunol. 1997 15:675-705)

Human CCR5 is composed of 352 amino acids with an intra-cellular C-terminus containing structural motifs for G-protein association and ligand-dependent signaling (M. Oppermann Cellular Signaling 2004 16:1201-1210). The extracellular N-terminal domain contributes to high-affinity chemokine binding and interactions with the gp120 HIV-1 protein (T. Dragic J. Gen. Virol. 2001 82:1807-1814; C. Blanpain et al. J. Biol. Chem. 1999 274:34719-34727). The binding site for the natural agonist RANTES (Regulated upon Activation and is Normal T-cell Expressed and Secreted) has been shown to be on the N-terminal domain and HIV-1 gp120 has been suggested to interact initially with the N-terminal domain and also with the ECL2 (B. Lee, et al. J. Biol. Chem. 1999 274:9617-26).

Modulators of the CCR5 receptor may be useful in the treatment of various inflammatory diseases and conditions, and in the treatment of infection by HIV-1 and genetically related retroviruses. As leukocyte chemotactic factors, chemokines play an indispensable role in the attraction of leukocytes to various tissues of the body, a process which is essential for both inflammation and the body's response to infection. Because chemokines and their receptors are central to the pathophysiology of inflammatory, autoimmune and infectious diseases, agents which are active in modulating, preferably antagonizing, the activity of chemokines and their receptors, are useful in the therapeutic treatment of these diseases. The CCR5 receptor is of particular importance in the context of treating inflammatory and infectious diseases. The natural ligands for CCR5 are the macrophage inflammatory proteins (MIP) designated MIP-1α and MIP-1b and RANTES.

HIV-1 infects cells of the monocyte-macrophage lineage and helper T-cell lymphocytes by exploiting a high affinity interaction of the viral enveloped glycoprotein (Env) with the CD4 antigen. The CD4 antigen, however appeared to be a necessary, but not sufficient, requirement for cell entry and at least one other surface protein was required to infect the cells (E. A. Berger et al., Ann. Rev. Immunol. 1999 17:657-700). Two chemokine receptors, either the CCR5 or the CXCR4 receptor were subsequently found to be co-receptors which are required, along with CD4, for infection of cells by the human immunodeficiency virus (HIV-1). The central role of CCR5 in the pathogenesis of HIV-1 was inferred by epidemiological identification of powerful disease modifying effects of the naturally occurring null allele CCR5 Δ32. The Δ32 mutation has a 32-base pair deletion in the CCR5 gene resulting in a truncated protein designated Δ32. Relative to the general population, Δ32/Δ32 homozygotes are significantly common in exposed/uninfected individuals suggesting the role of CCR5 in HIV-1 cell entry (R. Liu et al., Cell 1996 86(3):367-377; M. Samson et al., Nature 1996 382(6593):722-725).

The HIV-1 envelope protein is comprised of two subunits: gp120, the surface subunit and gp41, the transmembrane subunit. The two subunits are non-covalently associated and form homotrimers which compose the HIV-1 envelope. Each gp41 subunit contains two helical heptad repeat regions, HR1 and HR2 and a hydrophobic fusion region on the C-terminus.

The CD4 binding site on the gp120 of HIV-1 appears to interact with the CD4 molecule on the cell surface inducing a conformation change in gp120 which creates or exposes a cryptic CCR5 (or CXCR4) binding site, and undergoes conformational changes which permits binding of gp120 to the CCR5 and/or CXCR4 cell-surface receptor. The bivalent interaction brings the virus membrane into close proximity with the target cell membrane and the hydrophobic fusion region can insert into the target cell membrane. A conformation change in gp41 creates a contact between the outer leaflet of the target cell membrane and the viral membrane which produces a fusion pore whereby viral core containing genomic RNA enters the cytoplasm.

Viral fusion and cell entry is a complex multi-step process and each step affords the potential for therapeutic intervention. These steps include (i) CD40-gp120 interactions, (ii) CCR5 and/or CXCR-4 interactions and (iii) gp41 mediated membrane fusion. Conformational changes induced by these steps expose additional targets for chemotherapeutic intervention. Each of these steps affords an opportunity for therapeutic intervention in preventing or slowing HIV-1 infection. Small molecules (Q. Guo et al. J. Virol. 2003 77:10528-63) and antibodies (D. R. Kuritzkes et al. 10^{th} Conference on Retroviruses and Opportunistic Infections, Feb. 10-14, 2003, Boston, Mass. Abstract 13; K. A. Nagashima et al. J. Infect. Dis. 2001 183:1121-25) designed to prevent the gp120/CD4 interaction have been disclosed. Small molecule antagonists of, and antibodies to, CCR5 are discussed below. A small molecular weight antagonist of CXCR4 has been explored (J. Blanco et al. Antimicrob. Agents Chemother. 2000 46:1336-39). Enfuvirtide (T20, ENF or FUZEON®) is a 36 amino acid peptide corresponding to residues 643-678 in the HR2 domain of gp41. Enfuvirtide binds to the trimeric coiled-coil by the HR1 domains and acts in a dominant negative manner to block the endogenous six helix bundle formation thus inhibiting viral fusion. (J. M. Kilby et al., New Eng J. Med. 1998 4(11): 1302-1307). Enfuvirtide has been approved for clinical use.

In addition to the potential for CCR5 modulators in the management of HIV-1 infections, the CCR5 receptor is an important regulator of immune function and compounds of the present invention may prove valuable in the treatment of disorders of the immune system. Treatment of solid organ transplant rejection, graft v. host disease, arthritis, rheumatoid arthritis, inflammatory bowel disease, atopic dermatitis, psoriasis, asthma, allergies or multiple sclerosis by administering to a human in need of such treatment an effective amount of a CCR5 antagonist compound of the present invention is also possible.

### SUMMARY OF THE INVENTION

The present invention relates to pharmaceutical compositions for treating an HIV-1 infection, or preventing an HIV-1 infection, or treating AIDS or ARC, comprising co-administering a therapeutically effective amount of a synergistic combination of an isolated antibody which antibody binds to the CCR5 receptor and wherein the CDR3 of the variable heavy chain amino acid sequence of said antibody is selected from the group consisting of SEQ ID NO. 9 or 10, along with a CCR5 antagonist, a viral fusion inhibitor or a viral attachment inhibitor.

### BRIEF DESCRIPTION OF FIGURES

FIG. 1—depicts the structures of representative low molecular weight antagonists of the CCR5 receptor which are synergistic in combination with monoclonal antibodies RoAb13 and RoAb14.

FIG. 2—(A) depicts the synergistic interaction between RoAb14 and MVC in the cell-cell fusion as response surface utilizing the Greco Model. RoAb14 was added serially from 0 to 65 nM and MVC was added from 0 to 200 nM. The doses of both inhibitors are plotted against percent synergy. Percent synergy at each 10% increment is differentially shaded. (B) Isobologram of RoAb14-MVC combination plotted at the 95% inhibition level.

FIG. 3—Dose-response surface for RoAb13-MVC combinations. Percent synergy obtained from each combination dose was plotted against RoAb13 and MVC doses utilizing the Greco (A) and Prichard (B) models.

FIG. 4—The graph illustrates the effect of CCR5 antagonists on the time course of 5 mAb binding. CHO—CCR5 cells were pre-incubated with 50 nM of AK602, MVC, SCH-D, or vehicle at RT for 1 h, then incubated with CCR5 mAb ROAb14 (A), ROAb13 (B), 2D7 (C), or 45523 (D) at 0° C. for various time points, followed by cell fixation in 2% paraformaldehyde and FACS (Fluorescent Activated Cell Sorting) analysis. The time course curves for each mAb in the presence of various antagonists were created based on their mean fluorescence intensity (MFI) values.

FIG. 5—The graph illustrates the effect of CCR5 mAbs on MVC binding to CHO—CCR5 cells. The cells (2×10⁵/100 μL) were pre-incubated with 30 μg/mL of CCR5 mAbs or PBS at RT for 1 h, then incubated with 26 nM of ³H-MVC. At the end of various time points, cells were washed and the membrane bound ³H-MVC was measured as described in Example 2. The maximal counts from the control samples were set as 100% binding and the relative binding for all other samples were calculated and the time course curves were generated based on these relative binding at each time point.

### DETAILED DESCRIPTION OF THE INVENTION

In one embodiment of the present invention there is provided a pharmaceutical composition for treating an HIV-1 infection, or preventing an HIV-1 infection, or treating AIDS or ARC, comprising a therapeutically effective amount of a synergistic combination of an isolated antibody which antibody binds to the CCR5 receptor and wherein the CDR3 of the variable heavy chain amino acid sequence of said antibody is either SEQ ID NO. 9 or 10, and of a CCR5 antagonist, a viral fusion inhibitor or a viral attachment inhibitor.

In another embodiment of the present invention there is provided a pharmaceutical composition comprising a synergistic combination of an isolated antibody which antibody binds to the CCR5 receptor and wherein the CDR3 of the variable heavy chain amino acid sequence of said antibody is either SEQ ID NO. 9 or 10, and at least one additional antiviral agent selected from enfuviritide, TNX-355, TAK-220, TAK-779, AK602(ONO 4128), SCH—C, SCH-D, MVC, and a compound according to formula Ia-Id wherein R¹, R², R³ and Ar are as defined in claim 2.

In another embodiment of the present invention there is provided a pharmaceutical composition comprising a synergistic combination of an isolated antibody which antibody binds to the CCR5 receptor and wherein the CDR3 of the variable heavy chain amino acid sequence of said antibody is either SEQ ID NO. 9 or 10, and at least one additional CCR5 antagonist disclosed in WO2005075484 or in WO2005121145 both of which are hereby incorporated by reference in their entirety.

In another embodiment of the present invention there is provided a pharmaceutical composition comprising a synergistic combination of an isolated antibody which antibody binds to the CCR5 receptor and wherein the CDR3 of the variable heavy chain amino acid sequence of said antibody is either SEQ ID NO. 9 or 10, and at least one additional CCR5 antagonist selected from I-1 to I-22 in TABLE 1.

In another embodiment of the present invention there is provided a pharmaceutical composition comprising a therapeutically effective amount of a synergistic combination comprising an isolated antibody to the CCR5 receptor wherein the heavy and light variable domains are (i) SEQ ID NO: 1 and SEQ ID NO: 2; (ii) SEQ ID NO: 3 and SEQ ID NO: 4; (iii) SEQ ID NO: 5 and SEQ ID NO: 6 or (iv) SEQ ID NO: 7 and SEQ ID NO: 8 and a CCR5 antagonist, a viral fusion inhibitor or a viral attachment inhibitor.

In another embodiment of the present invention there is provided a pharmaceutical composition comprising a therapeutically effective amount of a synergistic combination comprising an isolated antibody to the CCR5 receptor wherein the heavy and light variable domains are (i) SEQ ID NO: 1 and SEQ ID NO: 2; (ii) SEQ ID NO: 3 and SEQ ID NO: 4; (iii) SEQ ID NO: 5 and SEQ ID NO: 6 or (iv) SEQ ID NO: 7 and SEQ ID NO: 8 and at least one CCR5 antagonist selected from TAK-220, TAK-779, AK602(ONO 4128), SCH—C, SCH-D, MVC or a compound according to formula Ia-Id wherein Ar, R¹, R² and R³ are as defined in claim 2.

In another embodiment of the present invention there is provided a pharmaceutical composition comprising a therapeutically effective amount of a synergistic combination comprising an isolated antibody to the CCR5 receptor wherein the heavy and light variable domains are (i) SEQ ID NO: 1 and SEQ ID NO: 2; (ii) SEQ ID NO: 3 and SEQ ID NO: 4; (iii) SEQ ID NO: 5 and SEQ ID NO: 6 or (iv) SEQ ID NO: 7 and SEQ ID NO: 8 and at least one additional CCR5 antagonist disclosed in WO2005075484 or in WO2005121145

In another embodiment of the present invention there is provided a pharmaceutical composition comprising a therapeutically effective amount of a synergistic combination comprising an isolated antibody to the CCR5 receptor wherein the heavy and light variable domains are (i) SEQ ID NO: 1 and SEQ ID NO: 2; (ii) SEQ ID NO: 3 and SEQ ID NO: 4; (iii) SEQ ID NO: 5 and SEQ ID NO: 6 or (iv) SEQ ID NO: 7 and SEQ ID NO: 8 and enfuviritide.

In another embodiment of the present invention there is provided a pharmaceutical composition comprising a therapeutically effective amount of a synergistic combination comprising an isolated antibody to the CCR5 receptor wherein the heavy and light variable domains are (i) SEQ ID NO: 1 and SEQ ID NO: 2; (ii) SEQ ID NO: 3 and SEQ ID NO: 4; (iii) SEQ ID NO: 5 and SEQ ID NO: 6 or (iv) SEQ ID NO: 7 and SEQ ID NO: 8 and the CD4 antibody TNX-355.

In another embodiment of the present invention there is provided a pharmaceutical composition comprising a therapeutically effective amount of a synergistic combination of an isolated antibody produced by a hybridoma cell line selected from m<CCR5>Pz01.F3, m<CCR5>Px04.F6, m<CCR5>Pz03.1C5 or m<CCR5>Px02.1C11 along with a CCR5 antagonist, a viral fusion inhibitor or a viral attachment inhibitor.

In another embodiment of the present invention there is provided a method for treating an HIV-1 infection, or preventing an HIV-1 infection, or treating AIDS or ARC, comprising co-administering to a host in need thereof a therapeutically effective amount of a synergistic combination of an isolated antibody which antibody binds to the CCR5 receptor and wherein the CDR3 of the variable heavy chain amino acid sequence of said antibody is either SEQ ID NO. 9 or 10, and a CCR5 antagonist, a viral fusion inhibitor or a viral attachment inhibitor.

In another embodiment of the present invention there is provided a method comprising co-administering to a host in need thereof a therapeutically effective amount of a synergistic combination of an isolated antibody which antibody binds to the CCR5 receptor and wherein the CDR3 of the variable heavy chain amino acid sequence of said antibody is either SEQ ID NO. 9 or 10, along with TAK-220, TAK-779, AK602(ONO 4128), SCH—C, SCH-D, MVC and a compound according to formula Ia-Id wherein Ar, R¹, R² and R³ are as defined in claim 2.

In another embodiment of the present invention there is provided a method comprising co-administering to a host in need thereof a therapeutically effective amount of a synergistic combination of an isolated antibody which antibody binds to the CCR5 receptor and wherein the CDR3 of the variable heavy chain amino acid sequence of said antibody is either SEQ ID NO. 9 or 10, and enfuviritide.

In another embodiment of the present invention there is provided a method comprising co-administering to a host in need thereof a therapeutically effective amount of a synergistic combination of an isolated antibody which antibody binds to the CCR5 receptor and wherein the CDR3 of the variable heavy chain amino acid sequence of said antibody is either SEQ ID NO. 9 or 10, and TNX-355.

In another embodiment of the present invention there is provided a method for treating an HIV-1 infection, or preventing an HIV-1 infection, or treating AIDS or ARC, comprising co-administering to a host in need thereof a therapeutically effective amount of a synergistic combination of an isolated antibody to the CCR5 receptor wherein the heavy and light variable domains are (i) SEQ ID NO: 1 and SEQ ID NO: 2; (ii) SEQ ID NO: 3 and SEQ ID NO: 4; (iii) SEQ ID NO: 5 and SEQ ID NO: 6 or (iv) SEQ ID NO: 7 and SEQ ID NO: 8 and a CCR5 antagonist, a viral fusion inhibitor or a viral attachment inhibitor.

In another embodiment of the present invention there is provided a method for treating an HIV-1 infection, or preventing an HIV-1 infection, or treating AIDS or ARC, comprising co-administering to a host in need thereof a therapeutically effective amount of a synergistic combination of an isolated antibody produced by a hybridoma cell line selected from m<CCR5>Pz01.F3, m<CCR5>Px04.F6, m<CCR5>Pz03.1C5 or m<CCR5>Px02.1C11 and a CCR5 antagonist, a viral fusion inhibitor or a viral attachment inhibitor.

In another embodiment of the present invention there is provided a method for treating an HIV-1 infection, or preventing an HIV-1 infection, or treating AIDS or ARC, comprising co-administering to a host in need thereof a therapeutically effective amount of a synergistic combination of an isolated antibody produced by a hybridoma cell line selected from m<CCR5>Pz01.F3, m<CCR5>Px04.F6, m<CCR5>Pz03.1C5 or m<CCR5>Px02.1C11 and a CCR5 antagonist is selected from the group consisting of TAK-220, TAK-779, AK602(ONO 4128), SCH—C, SCH-D, MVC and a compound according to formula Ia-Id wherein Ar, R¹, R² and R³ are as defined in claim 2.

In another embodiment of the present invention there is provided a method for treating an HIV-1 infection, or preventing an HIV-1 infection, or treating AIDS or ARC, comprising co-administering to a host in need thereof a therapeutically effective amount of a synergistic combination of an isolated antibody produced by a hybridoma cell line selected from m<CCR5>Pz01.F3, m<CCR5>Px04.F6, m<CCR5>Pz03.1C5 or m<CCR5>Px02.1C11 and enfuviritide.

In another embodiment of the present invention there is provided a method for treating an HIV-1 infection, or preventing an HIV-1 infection, or treating AIDS or ARC, comprising co-administering to a host in need thereof a therapeutically effective amount of a synergistic combination of an isolated antibody produced by a hybridoma cell line selected from m<CCR5>Pz01.F3, m<CCR5>Px04.F6, m<CCR5>Pz03.1C5 or m<CCR5>Px02.1C11 and TNX-335.

### DEFINITIONS

The term “CCR5” as used herein refers to a chemokine receptor which binds members of the C—C group of chemokines and whose amino acid sequence comprises that provided in Genbank Accession Number 1705896 and related polymorphic structures. The term “chemokine” refers to a cytokine that can stimulate leukocyte movement. Since the CCR5 receptor has been identified as a co-receptor along with CD4 for HIV-1 cell entry by macrophage-tropic (M-tropic) strains of HIV-1, it has become a target for chemotherapy. Both traditional small molecule approaches and macromolecular approaches to inhibition of HIV fusion have been disclosed.

The term “antibody” (Ab) as used herein includes monoclonal antibodies (mAb), polyclonal antibodies antibody fragments sufficiently long to exhibit the desired biological activity. The term “immunoglobulin” (Ig) is used interchangeably with “antibody” herein. An “isolated antibody” is one which has been identified and separated and/or recovered from a component of its natural environment or from the cell in which it was produced. Contaminant components of its natural environment are materials which would interfere with therapeutic uses for the antibody, and may include enzymes, hormones, and other proteinaceous or nonproteinaceous solutes.

The basic 4-chain antibody unit of an IgG antibody is a heterotetrameric glycoprotein composed of two identical light (L) chains and two identical heavy (H) chains. The 4-chain unit of an IgG antibody is generally about 150,000 daltons. Each L chain is linked to an H chain by one disulfide bond, while the two H chains are linked to each other by one or more disulfide bonds depending on the H chain isotype. Each H and L chain also has regularly spaced intrachain disulfide bridges. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H} 1). The L chain from any vertebrate species can be assigned to one of two clearly distinct types, called kappa and lambda, based on the amino acid sequences of their constant domains. Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5^{th} Ed. Public Health Service, National Institutes of Health, Bethesda, Md. 1991). The constant domains are not involved directly in binding an antibody to an antigen, but exhibit various effector functions.

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 1-35 (H1), 50-65 (H2) and 95-102 (H3) in the V_{H}; Kabat et al., supra) and/or those residues from a “hypervariable loop” (e.g. residues 26-32 (L1), 50-52 (L2) and 91-96 (L3) in the V_{L}, and 26-32 (H1), 53-55 (H2) and 96-101 (H3) in the V_{H}; Chothia and Lesk, J. Mol. Biol. 1987 196:901-917).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site (epitope) unlike polyclonal antibody preparations which include different antibodies directed against different epitopes. Monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al. (Nature 1975 256:495), or may be made using recombinant DNA methods in bacterial, eukaryotic animal or plant cells (see, e.g., U.S. Pat. No. 4,816,567).

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA 1984 81:6851-6855). Chimeric antibodies of interest herein include “primitized” antibodies comprising variable domain antigen-binding sequences derived from a non-human primate (e.g. Old World Monkey, Ape etc.), and human constant region sequences. Chimeric antibodies are produced to reduce Human Anti-Murine Antibody (HAMA) responses elicited by murine antibodies. Generally, chimeric antibodies contain approximately 33% mouse protein in the variable region and 67% human protein in the constant region. Chimeric antibodies can exhibit a Human Anti-Chimeric Antibodies (ACA) response similar to the HAMA response which may limit their therapeutic potential. The use of chimeric antibodies substantially reduced the HAMA responses but did not eliminate them (K. Kuus-Reichel et al., Clin. Diagn Lab Immunol. 19941:365-372; M. V. Pimm Life Sci. 1994 55:PL45-PL49). Partially humanized antibodies then were developed in which the 6 CDRs of the heavy and light chains and a limited number of structural amino acids of the murine monoclonal antibody were grafted by recombinant technology to the CDR-depleted human IgG scaffold. (P. T. Jones et al., Nature 1986 321:522-525) Although this process further reduced or eliminated the HAMA responses, in many cases, substantial further antibody design procedures were needed to reestablish the required specificity and affinity of the original murine antibody. (J. D. Isaacs Rheumatology 2001 40:724-738)

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further optimize antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 1986 321:522-525; Riechmann et al., Nature 1988 332:323-329; and Presta, Curr. Opin. Struct. Biol. 1992 2:593-596.

The choice of human variable domains, both light and heavy, to be used in making the humanized antibodies is very important to reduce antigenicity and HAMA response when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (M. J. Sims et al., J. Immunol. 1993 151:2296; Chothia et al., J. Mol. Biol. 1987 196:901). Another method uses a particular framework region derived from the consensus sequence of all human antibodies of a particular subgroup of light or heavy chains.

An alternate approach is to replace the immunogenic-epitopes in the murine variable domains with benign amino acid sequences to produce a deimmunized variable domain. The deimmunized variable domains are linked genetically to human IgG constant domains to yield a deimmunized antibody. The term “deimmunized antibody” as used herein refers an antibody which has been modified to replace immunogenic epitopes in a murine variable domain with non-immunogenic amino acid sequences. The deimmunized variable domains are linked to a human Fc domain by recombinant techniques. Deimmunized sequences are identified using computerized docking protocols to identify segments of the antibody which may bind to class II MHC complex. Amino acid substitutions are made to abolish MHC presentation, ideally without alteration of specificity and affinity for then epitope; however, further modifications may be made to optimize the binding. Deimmunized antibodies resulting from these modifications which do not alter the epitope specificity are contemplated as within the scope of the invention.

The phrase “natural effector functions” as used herein refers to antigen elimination processes mediated by immunoglobulins and initiated by binding of the effector molecules to the Fc segment of the antibody. The common effector functions include complement-dependent cytotoxicity, phagocytosis and antibody-dependent cellular cytotoxicity.

An “intact” antibody is one which comprises an antigen-binding site as well as a C_{L} and at least heavy chain constant domains, C_{H}1, C_{H}2 and C_{H}3. The constant domains may be native sequence constant domains (e.g. human native sequence constant domains) or an amino acid sequence variant thereof.

An “antibody fragment” comprises a portion of an intact antibody, preferably the antigen binding or variable region of the intact antibody. Examples of antibody fragments include Fab, Fab′, F(ab′)₂, and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI.

Papain digestion of antibodies produces two identical antigen-binding fragments, called “Fab” fragments, and a residual “Fc” fragment, a designation reflecting the ability to crystallize readily. The Fc fragment comprises the carboxy-terminal portions of both H chains held together by disulfides. The effector functions of antibodies are determined by sequences in the Fc region, which is also the fragment recognized by Fc receptors (FcR) found on certain types of cells. The Fab fragment consists of an entire L chain along with the variable region domain of the H chain (V_{H}), and the first constant domain of one heavy chain (C_{H} 1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H} 1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “amino acid sequence variant” refers to a polypeptide that has amino acid sequences that differ to some extent from a native sequence polypeptide. The amino acid sequence variants can possess substitutions, deletions, and/or insertions at certain positions within the amino acid sequence of the native amino acid sequence. “Homology” is defined as the percentage of residues in the amino acid sequence variant that are identical after aligning the sequences and introducing gaps, if necessary, to achieve the maximum percent homology. Methods and computer programs for the alignment are well known in the art. Sequence variants which do not alter the specificity or synergistic properties of the present invention are readily determined experimentally and fall within the scope of the invention.

The term “epitope” as used herein means a protein determinant capable of specific binding to an antibody. Epitopes usually consist of chemically active surface groupings of molecules such as amino acids or sugar side chains and usually have specific three-dimensional structural characteristics as well as specific charge characteristics. Conformational and non-conformational epitopes are distinguished in that the former, but not the latter, is lost in the presence of denaturating solvents.

The term “synergy” or “synergistic” as used herein means the combined effect of the compounds when used in combination is greater than the additive effects of the compounds when used individually. Quantitative methods of detecting the existence of synergism are described below.

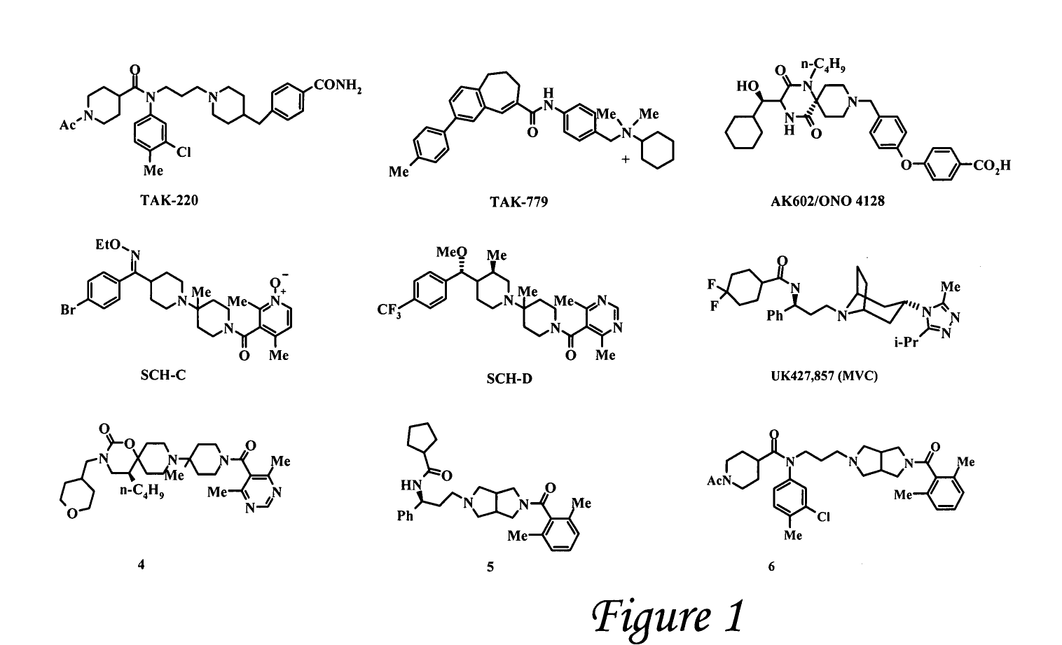

The recognition of the role of CCR5 and CXCR4 co-receptors in HIV-1 pathogenesis afforded new targets for intervention and programs to identify inhibitors of chemokine or gp120 binding were initiated. The interaction between viral envelope proteins and both the CD4 and chemokine co-receptors is complex and affords multiple opportunities for chemotherapeutic intervention. The efforts to identify chemokine modulators have been reviewed. (F. Shaheen and R. G. Collman, Cur. Opin. Infect Dis. 2004, 17:7-16; W. Kazmierski et al. Biorg Med. Chem. 2003 11:2663-76; L. Agrawal and G. Alkhatib, Expert Opin. Ther. Targets 2001 5(3):303-326; Chemokine CCR5 antagonists incorporating 4-aminopiperidine scaffold, Expert Opin. Ther. Patents 2003 13(9):1469-1473; M. A. Cascieri and M. S. Springer, Curr. Opin. Chem. Biol. 2000 4:420-426, and references cited therein) Both small molecule CCR5 antagonists and macromolecular antibodies have been investigated. Representative low molecular-weight CCR5 antagonists are depicted in FIG. 1 and IC₅₀s in a cell-cell fusion assay are tabulated in TABLE 2. All CCR5 antagonists exhibited low nM or sub-nanomolar IC₅₀s (0.4-5 nM) in the CCF assay system.

### Low Molecular-Weight CCR5Antagonists

Takeda's identified TAK-779 as a potential CCR5 antagonist. (M. Shiraishi et al., J. Med. Chem. 2000 43(10):2049-2063; M. Babba et al. Proc. Nat. Acad. Sci. USA 1999 96:5698-5703) and TAK-220 (C. Tremblay et al. Antimicrob. Agents Chemother. 2005 49(8):3483-3485). TAK-220 has been shown to interact with Asn252 and L225 in TM6 along with G163 and I198 in TMs 4 and 5, respectively (M. Nishikawa et al. Antimicrob. Agent Chemother. 2005 49(11):4708-4715). An analysis of TAK-779 binding to Ala mutants suggested residues L33, Y37, T82, W86, Y108 and T123 located on TMs 1, 2, 3, and 7 are important residues which interact with the antagonist. (T. Drajic et al. Proc. Nat. Acad. Sci. USA 2000 97(10):5639-5644)

WO0039125 (D. R. Armour et al.) and WO0190106 (M. Perros et al.) disclose heterocyclic compounds that are potent and selective CCR5 antagonists. Pfizer's UK-427,857 (MVC) has advanced to phase III clinical trials and show activity against HIV-1 isolates and laboratory strains (P. Dorr et al., Antimicrob. Agents Chemother. 2005 49(11):4721-4732; A. Wood and D. Armour, Prog. Med. Chem. 2005 43:239-271; C. Watson et al., Mol. Pharm. 2005 67(4):1268-1282; M. J. Macartney et al., 43^{rd} Intersci. Conf. Antimicrob. Agents Chemother. Sep. 14-17, 2003, Abstract H-875).

Schering has advanced Sch-351125 (SCH—C) into Phase I/II clinical studies and reported the advance of a more potent follow-up compound, Sch-417690 (SCH-D) into Phase I studies. (S. W. McCrombie et al., WO00066559; B. M. Baroudy et al. WO00066558; A. Palani et al., J. Med. Chem. 2001 44(21):3339-3342; J. R. Tagat et al., J. Med. Chem. 2001 44(21):3343-3346; J. A. Esté, Cur. Opin. Invest. Drugs 2002 3(3):379-383; J. M. Struzki et al. Proc. Nat. Acad. Sci. USA 2001 98:12718-12723). Modeling studies with alanine mutants and SCH C suggested the activity was dependent on residues in transmembrane helices 1, 2, 3, 5 and 7 and in particular L33 and Y37 (TM1), D76 and W86 (TM2), F113 (TM3), I198 (TM5) and E283 (TM6)(F. Tsamis et al. J. Virol. 2003 77(9):5201-5208).

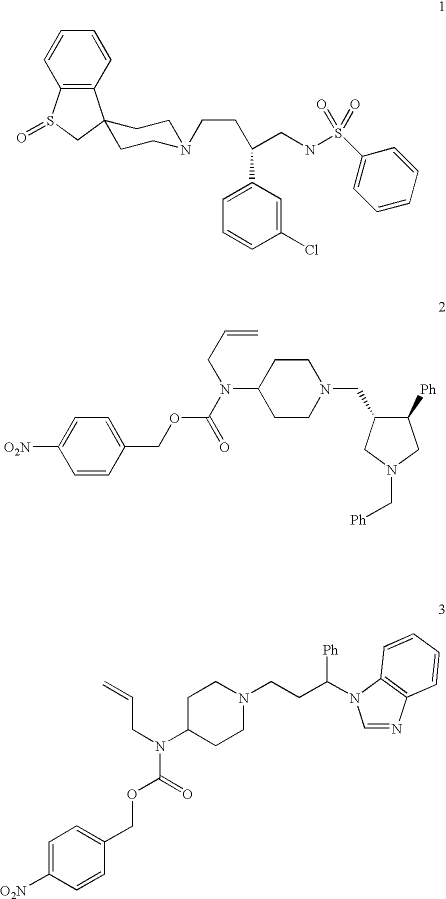

Merck has disclosed the preparation of (2S)-2-(3-chlorophenyl)-1-N-(methyl)-N-(phenylsulfonyl)amino]-4-[spiro(2,3-dihydrobenzothiophene-3,4′-piperidin-1′-yl)butane S-oxide (1) and related derivatives, trisubstituted pyrrolidines 2 and substituted piperidines 3 with good affinity for the CCR5 receptor and potent-HIV-1 activity. (P. E. Finke et al., Bioorg. Med. Chem. Lett., 2001 11:265-270; P. E. Finke et al., Bioorg. Med. Chem. Lett., 2001 11:2469-2475; P. E. Finke et al., Bioorg Med. Chem. Lett., 2001 11:2475-2479; J. J. Hale et al., Bioorg. Med. Chem. Lett., 2001 11:2741-22745; D. Kim et al., Bioorg. Med. Chem. Lett., 2001 11:3099-3102) C. L. Lynch et al. Org. Lett. 2003 5:2473-2475; R. S. Veazey et al. J. Exp. Med. 2003198:1551-1562.

ONO-4128, E-913, AK-602 was identified in a program initiated at Kumamoto University (K. Maeda et al. J. Biol. Chem. 2001 276:35194-35200; H. Nakata et al. J. Virol. 2005 79(4):2087-2096)

In WO00/166525; WO00/187839; WO02/076948; WO02/076948; WO02/079156, WO2002070749, WO2003080574, WO2003042178, WO2004056773, WO2004018425 Astra Zeneca disclose 4-amino piperidine compounds which are CCR5 antagonists.

Other representative CCR5 antagonists which could be used in synergistic compositions with an antibody or useful for treating HIV-1 infections as disclosed herein include compounds according to formula Ia-Id

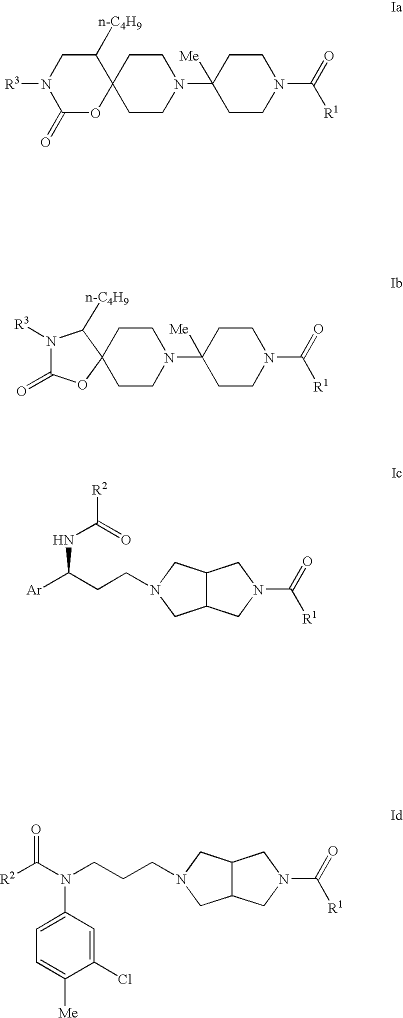

wherein
Ar is phenyl, 3-fluorophenyl, 3-chlorophenyl or 3,5-difluorophenyl;
R¹ is selected from the group consisting of:

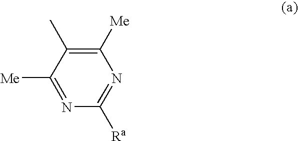

wherein R^{a} is hydrogen, —OH, —NMeCH₂CONH₂ or —OCMe₂CONH₂;

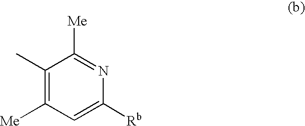

wherein R^{b} is hydrogen or cyano;

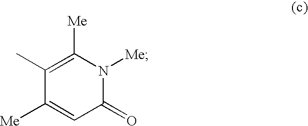

and,

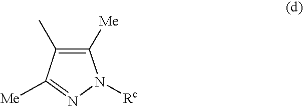

wherein R^{c} is 6-trifluoromethylpyridazin-3-yl, pyrimidin-5-yl, 5-trifluoromethyl-pyridin-2-yl;
R² is selected from the group consisting of cyclopentyl, 2-carboxy-cyclopentyl, 3-oxo-cyclopentyl, 3-oxo-cyclohexyl, 3-oxo-cyclobutyl, 3-oxa-cyclopentyl, 2-oxa-cyclopentyl, 4,4-difluorocyclohexyl, 3,3-difluoro-cyclobutyl, N-acetyl-azetidin-3-yl, N-methylsulfonyl-azetidin-3-yl and methoxycarbonyl;
R³ is selected from the group consisting of cyclohexyl methyl, tetrahydro-pyran-4-yl methyl; 4-methoxy-cyclohexanyl, 4-fluoro-benzyl, 4,4-difluorocyclohexyl-methyl, 2-morpholin-4-yl-ethyl and N—C₁₋₃ alkoxycarbonyl-piperidin-4-yl methyl; and,
   pharmaceutically acceptable salts thereof.

Compounds according to formula Ia and Ib have been disclosed by S. M Gabriel and D. M. Rotstein in WO2005075484 published Aug. 18, 2005. Compounds according to formula Ic and Id have been disclosed by E. K. Lee et al. in WO2005121145 published Dec. 22, 2005. The compositions and methods herein disclosed can be practiced with the compounds disclosed therein. Some particular compounds according to formula Ia-Id are tabulated to TABLE 1. In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight.

One skilled in the art will realize that many other analogs of similar structure have been prepared and their use in compositions containing an anti-CCR5 antibody is within the scope of the present case and therefore this TABLE is not intended to be limiting. The search for CCR5 antagonists us an active area of research and new structures will certainly be identified which will form synergistic combinations with the mAbs herein described. One skilled in the art will appreciate that the level of synergism can be determined without undue experimentation and such combinations are within the scope of the current claims.

TABLE 1

| | |
|---|---|
| I-1 | 4,4-Difluoro-cyclohexanecarboxylic acid {(S)-3-[5-(4,6-dimethyl-pyrimidine-5-carbonyl)- |
| | hexahydro-pyrrolo[3,4-c]pyrrol-2-yl]-1-phenyl-propyl}-amide |
| I-2 | (S)-5-Butyl-9-[1-(4,6-dimethyl-pyrimidine-5-carbonyl)-4-methyl-piperidin-4-yl]-3-(tetrahydro- |
| | pyran-4-ylmethyl)-1-oxa-3,9-diaza-spiro[5.5]undecan-2-one |
| I-3 | 3,3-Difluoro-cyclobutanecarboxylic acid [(S)-3-[5-(4,6-dimethyl-pyrimidine-5-carbonyl)- |
| | hexahydro-pyrrolo[3,4-c]pyrrol-2-yl]-1-(3-fluoro-phenyl)-propyl]-amide |
| I-4 | 5-Butyl-9-[1-(4,6-dimethyl-pyrimidine-5-carbonyl)-4-methyl-piperidin-4-yl]-3-(4-methoxy- |
| | cyclohexylmethyl)-1-oxa-3,9-diaza-spiro[5.5]undecan-2-one |
| I-5 | 1-Acetyl-azetidine-3-carboxylic acid {(S)-3-[5-(2,4-dimethyl-pyridine-3-carbonyl)-hexahydro- |
| | pyrrolo[3,4-c]pyrrol-2-yl]-1-phenyl-propyl}-amide |
| I-6 | 3,3-Difluoro-cyclobutanecarboxylic acid {(S)-1-(3-chloro-phenyl)-3-[5-(4,6-dimethyl- |
| | pyrimidine-5-carbonyl)-hexahydro-pyrrolo[3,4-c]pyrrol-2-yl]-propyl}-amide |
| I-7 | 3-Hydroxy-cyclopentanecarboxylic acid [(S)-3-[5-(2,4-dimethyl-pyridine-3-carbonyl)- |
| | hexahydro-pyrrolo[3,4-c]pyrrol-2-yl]-1-(3-fluoro-phenyl)-propyl]-amide; compound with |
| | trifluoro-acetic acid |
| I-8 | 2-Hydroxy-cyclopentanecarboxylic acid [(S)-3-[5-(4,6-dimethyl-pyrimidine-5-carbonyl)- |
| | hexahydro-pyrrolo[3,4-c]pyrrol-2-yl]-1-(3-fluoro-phenyl)-propyl]-amide; compound with |
| | trifluoro-acetic acid |
| I-9 | Cyclopentanecarboxylic acid [(S)-3-[5-(3,5-dimethyl-1-pyrimidin-5-yl-1H-pyrazole-4- |
| | carbonyl)-hexahydro-pyrrolo[3,4-c]pyrrol-2-yl]-1-(3-fluoro-phenyl)-propyl]-amide |
| I-10 | 3-Oxo-cyclobutanecarboxylic acid [(S)-3-[5-(6-cyano-2,4-dimethyl-pyridine-3-carbonyl)- |
| | hexahydro-pyrrolo[3,4-c]pyrrol-2-yl]-1-(3-fluoro-phenyl)-propyl]-amide |
| I-11 | 5-Butyl-9-[1-(4,6-dimethyl-pyrimidine-5-carbonyl)-4-methyl-piperidin-4-yl]-3-[2-(tetrahydro- |
| | pyran-4-yl)-ethyl]-1-oxa-3,9-diaza-spiro[5.5]undecan-2-one |
| I-12 | 3,3-Difluoro-cyclobutanecarboxylic acid {(S)-1-phenyl-3-[5-(1,2,4-trimethyl-6-oxo-1,6- |
| | dihydro-pyridine-3-carbonyl)-hexahydro-pyrrolo[3,4-c]pyrrol-2-yl]-propyl}-amide |
| I-13 | 1-Acetyl-azetidine-3-carboxylic acid {(S)-3-[5-(6-cyano-2,4-dimethyl-pyridine-3-carbonyl)- |
| | hexahydro-pyrrolo[3,4-c]pyrrol-2-yl]-1-phenyl-propyl}-amide |
| I-14 | 1-Methanesulfonyl-azetidine-3-carboxylic acid {(S)-3-[5-(4,6-dimethyl-pyrimidine-5- |
| | carbonyl)-hexahydro-pyrrolo[3,4-c]pyrrol-2-yl]-1-phenyl-propyl}-amide; compound with |
| | trifluoro-acetic acid |
| I-15 | 4-Butyl-8-[1-(4,6-dimethyl-pyrimidine-5-carbonyl)-4-methyl-piperidin-4-yl]-3-(tetrahydro- |
| | pyran-4-ylmethyl)-1-oxa-3,8-diaza-spiro[4.5]decan-2-one |
| I-16 | 5-Butyl-9-[1-(4,6-dimethyl-pyrimidine-5-carbonyl)-4-methyl-piperidin-4-yl]-3-(2-morpholin- |
| | 4-yl-ethyl)-1-oxa-3,9-diaza-spiro[5.5]undecan-2-one |
| I-17 | 5-Butyl-9-[1-(4,6-dimethyl-pyrimidine-5-carbonyl)-4-methyl-piperidin-4-yl]-3-(4-fluoro- |
| | benzyl)-1-oxa-3,9-diaza-spiro[5.5]undecan-2-one |
| I-18 | Cyclopentanecarboxylic acid {(S)-3-[5-(2,6-dimethyl-benzoyl)-hexahydro-pyrrolo[3,4- |
| | c]pyrrol-2-yl]-1-phenyl-propyl}-amide |
| I-19 | 1-Acetyl-piperidine-4-carboxylic acid(3-chloro-4-methyl-phenyl)-{3-[5-(2,6-dimethyl- |
| | benzoyl)-hexahydro-pyrrolo[3,4-c]pyrrol-2-yl]-propyl}-amide |
| I-20 | 1-{8-[(S)-3-Acetylamino-3-(3-fluoro-phenyl)-propyl]-8-aza-bicyclo[3.2.1]oct-3-yl}-2-methyl- |
| | 1,4,6,7-tetrahydro-imidazo[4,5-c]pyridine-5-carboxylic acid methyl ester |
| I-21 | N-{(S)-1-(3-Fluoro-phenyl)-3-[3-(5-isobutyryl-2-methyl-4,5,6,7-tetrahydro-imidazo[4,5- |
| | c]pyridin-3-yl)-8-aza-bicyclo[3.2.1]oct-8-yl]-propyl}-acetamide |
| I-22 | 4-{(S)-5-Butyl-9-[1-(4,6-dimethyl-pyrimidine-5-carbonyl)-4-methyl-piperidin-4-yl]-2-oxo-1- |
| | oxa-3,9-diaza-spiro[5.5]undec-3-ylmethyl}-piperidine-1-carboxylic acid methyl ester |

### Fusion Inhibitors

Enfuviritide (FUZEON®, T-20) is a unique fusion inhibitor which binds to the viral envelope protein gp41 after the viral coat proteins bid to CD4 and CCR5 and interferes with the association of the viral envelop proteins and the host cell membrane. Enfuviritide is a 36 amino acid polypeptide which corresponds to residues 643-678 of HIV-1 gp160. Enfuviritide selectively inhibits HIV-1 cell fusion and does not inhibit cell fusion of HIV-2 or simian immunodeficiency virus. Enfuviritide is effective against viral strains resistant to other anti-retroviral drugs. (T. Matthews et al. Nat. Rev. Drug Discov. 2004 3:215-225)

### Attachment Inhibitors

TNX-355 is a humanized IgG4 monoclonal antibody that binds to a conformational epitope on domain 2 of CD4. (L. C. Burkly et al., J. Immunol. 1992 149:1779-87) The TNX-355 epitope becomes accessible after a conformational change induced by gp120/CD4 binding and therefore has no interaction with immune cells in the absence of HIV-1. TNX-355 can inhibit viral attachment of CCR5-, CXCR4- and dual/mixed tropic HIV-1 strains. (E. Godofsky et al., In Vitro Activity of the Humanized Anti-CD4 Monoclonal Antibody, TNX-355, against CCR5, CXCR4, and Dual-Tropic Isolates and Synergy with Enfuvirtide, 45th Annual Interscience Conference on Antimicrobial Agents and Chemotherapy (ICAAC). Dec. 16-19, 2005, Washington D.C. Abstract # 3844; D. Norris et al. TNX-355 in Combination with Optimized Background Regime (OBR) Exhibits Greater Antiviral Activity than OBR Alone in HIV-Treatment Experienced Patients, 45th Annual Interscience Conference on Antimicrobial Agents and Chemotherapy (ICAAC). Dec. 16-19, 2005, Washington D.C. Abstract #4020.)

### Anti-CCR5 Antibodies

Macromolecular therapeutics including antibodies, soluble receptors and biologically active fragments thereof have become an increasingly important adjunct to conventional low molecular weight drugs. (O. H. Brekke and I. Sandlie Nature Review Drug Discov. 2003 2:52-62; A. M. Reichert Nature Biotech. 2001 19:819-821) Antibodies with high specificity and affinity can be targeted at extra-cellular proteins essential for viral cell fusion. CD4, CCR5 and CXCR4 have been targets for antibodies which inhibit viral fusion.

V. Roschke et al. (Characterization of a Panel of Novel Human Monoclonal Antibodies that Specifically Antagonize CCR5 and Block HIV-1 Entry, 44th Annual Interscience Conference on Antimicrobial Agents and Chemotherapy (ICAAC). Oct. 29, 2004, Washington D.C. Abstract #2871) have disclosed monoclonal antibodies which bind to the CCR5 receptor and inhibit HIV entry into cells expressing the CCR5 receptor. L. Wu and C. R MacKay in U.S. Ser. No. 09/870,932 filed May 30, 2001 disclose monoclonal antibodies 5C7 and 2D7 which bind to the CCR5 receptor in a manner capable of inhibiting HIV infection of a cell. W. C. Olsen et al. (J. Virol. 1999 73(5):4145-4155) disclose monoclonal antibodies capable of inhibiting (i) HIV-1 cell entry, (ii) HIV-1 envelope-mediated membrane fusion, (iii) gp120 binding to CCR5 and (iv) CC-chemokine activity. Synergism between the anti-CCR5 antibody Pro140 and a low molecular weight CCR5 antagonists have been disclosed by Murga et al. (3rd IAS Conference on HIV Pathogenesis and Treatment, Abstract TuOa.02.06. Jul. 24-27, 2005, Rio de Janeiro, Brazil)

Anti-CCR5 antibodies have been isolated which inhibit HIV-1 cell entry including: RoAb13 (<CCR5>Pz01.F3), RoAb14 (<CCR5>Px02.1C11), RoAb15 (<CCR5>Pz03.1C5), RoAb16 (<CCR5>F3.1H12.2E5) have been disclosed in EP05007138.0 filed Apr. 1, 2005 which is hereby incorporated by reference in its entirety. The cell lines have been deposited in the Deutsche Sammlung von Mikroorganismen und Zellkulturen GmbH (DMSZ; German Collection of Microorganisms and Cell Cultures) on Aug. 18, 2004 with the following deposition numbers: m<CCR5>Px01.F3 (DSM ACC 2681), m<CCR5>Pz02.1C11 (DSM ACC 2682), m<CCR5>Pz03.1C5 (DDSM ACC 2683) and m<CCR5>Pz04.1F6 (DSM ACC 2684).

The deposit was made under the provisions of the Budapest Treaty on the International Recognition of the Deposit of Microorganisms for the Purpose of Patent Procedure and Regulations thereunder (Budapest treaty). This assures the maintenance of viable cultures for 30 years from the date of deposit. The organisms will be made available to the public upon issuance of the pertinent U.S. patent or upon laying open to the public of any U.S. or foreign patent application, whichever comes first.

Generation of Mouse Anti-Human CCR5 Monoclonal Antibodies (mAbs)

Polyclonal antibodies are preferably raised in animals by multiple subcutaneous (sc) or intraperitoneal (ip) injections of the relevant antigen and an adjuvant. Immunologic adjuvants are agents that enhance specific immune responses to antigens. Adjuvants have diverse mechanisms of action and should be selected for use based on the route of administration and the type of immune response (antibody, cell-mediated, or mucosal immunity) that is desired for a particular vaccine. Anti-CCR5 antibodies were elicited by immunization of mice with CHO or L1.2 cells with a high level of CCR5 expression along with Freud's complete adjuvant (FCA). Animals were immunized initially with 10⁷ CCR5 expressing cells and FCA. Subsequently immunizations were boosted at 4-6 week intervals with CCR5 expressing cells and Freund's Incomplete Adjuvant.

Monoclonal antibodies may be made using the hybridoma method first described by Kohler et al. (Nature 1975 256:495), or may be made by recombinant DNA methods. Recombinant production of antibodies is well-known in the state of the art and described, for example, in the review articles of S.C. Makrides, Protein Expr. Purif 1999 17:183-202; S. Geisse et al., Protein Expr. Purif. 1996 8:271-282; R. J. Kaufman, Mol. Biotechnol. 2000 16:151-161; R. G. Werner, Drug Res. 1998 48:870-880.

Spleens from the immunized mice were harvested and fused with a myeloma cell line using a suitable fusing agent, such as polyethylene glycol, to form a hybridoma cell. (J. W. Goding. In Monoclonal Antibodies: Principles and Practice, 2^{nd} Ed; Academic Press: New York, 1986, pp. 59-103) The hybridoma cells thus prepared are seeded and grown in a suitable culture medium which medium preferably contains one or more substances that inhibit the growth or survival of the unfused, parental myeloma cells (also referred to as fusion partner).

The antibodies of the present invention can be conveniently prepared by recombinant DNA technology. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include: A. Skerra, Curr. Opin. Immunol. 1993 5:256-262 and Pluckthun, Immunol. Rev. 1992 130:151-188.

The DNA that encodes the antibody may be modified to produce chimeric or fusion antibody polypeptides, for example, by substituting human heavy chain and light chain constant domain (C_{H} and C_{L}) sequences (i.e. humanized or deimmunized antibodies) for the homologous murine sequences (U.S. Pat. No. 4,816,567; and Morrison, et al., Proc. Nat. Acad. Sci. USA, 1984 81:6851), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody.

The specificity of the antibody resides in the complementary defining regions (i.e., the hypervariable regions of the F_{ab} portion of the antibodies). Other portions of the antibody molecule can be altered without modifying the epitope selectivity and it is frequently desirable to modify other portions of the antibody molecule to modify or eliminate pharmacodynamic properties thereof. Numerous techniques have been identified to reduce adverse effects from the non-antigen binding portion of the antibody molecule including chimeric, humanized, and deimmunized antibodies. Reduction of the antigenicity of non-human derived antibodies permits multiple dosing and implementation of techniques to the extend serum half-life. The aforementioned approaches to improving the safety profile of the anti-CCR5 antibody can be employed without departing from the spirit of the invention. Antibodies with the CDRs of the RoAb13-RoAb16 but which have been modified to eliminate untoward effects are within the scope of the present invention.

One skilled in the art will appreciate that antibody fragments which comprise a portion of a full length antibody may also have the properties described herein. The antibody fragment will contain the variable region thereof or at least the antigen binding portion thereof and retain sufficient size and functional sites to inhibition of viral cell fusion will behave in the same manner as the full length antibody.

Both monoclonal antibodies recognizing extracellular segments of the CCR5 receptor and low molecular weight allosteric CCR5 antagonists have been demonstrated to inhibit viral cell fusion in diverse assays assessing viral entry. Monoclonal antibodies RoAb13 and RoAb14 whose epitopes are on the amino terminus and ECL2 and are both potent inhibitors of viral entry. Two other commercially available antibodies 2D7 and 45523 exhibited potent (IC₅₀=4.3 nM) and weak (IC₅₀=23 nM) activity respectively. Compounds 4-6 are CCR5 antagonists identified at Roche Palo Alto. SCH-D, MVC and AK-602 are other CCR5 antagonists in development as viral fusion inhibitors. (see FIG. 1)

TABLE 2
Potency of CCR5 mAbs and antagonists in Cell-Cell Fusion Assay

| Inhibitor | Class | Company | IC₅₀ ± SD (nM) |
|---|---|---|---|
| 2D7 | Murine mAb | Millenium | 4.3 ± 1.6 |
| 45523 | Murine mAb | Commercial | 23 ± 6.7 |
| RoAb13 | Murine mAb | Roche | 14 ± 3.7 |
| RoAb14 | Murine mAb | Roche | 1.3 ± 0.4 |
| 4 | Antagonist | Roche | 1 ± 0.2 |
| 5 | Antagonist | Roche | 4 ± 1.1 |
| 6 | Antagonist | Roche | 0.4 ± 0.2 |
| SCH-D | Antagonist | Schering-Plough | 5 ± 2.4 |
| MVC | Antagonist | Pfizer | 0.6 ± 0.4 |
| AK602 | Antagonist | GSK/Ono | 0.5 ± 0.3 |

The elucidation of CCR5 and CXCR4 as co-receptors along with CD4 for HIV-1 cell fusion has afforded new target sites for anti-HIV-1 chemotherapy which can be included in anti-HIV1 combinations. Synergy between antibodies and CCR5 antagonists would enhance their utility. Surprisingly, antibodies RoAb13-RoAb16 have now been found to exhibit potent synergistic inhibition of HIV-1 cell fusion when administered with CCR5 antagonists, viral entry inhibitors or viral attachment inhibitors. The synergy was observed with all the diverse allosteric CCR5 antagonists examined. Synergy also was found between the monoclonal antibodies and the fusion inhibitor enfuvirtide (T-20). One skilled in the art will appreciate that the problem solved herein is the identification of antibodies with an epitope which allows concurrent binding of the antibody and the CCR5 antagonists, viral entry inhibitors or viral attachment inhibitors.

### Synergy

Combinations of anti-retroviral drugs have proven to be an effective strategy to control HIV-1 replication. Soon after the utility AZT in HIV-1 chemotherapy was noted, it became apparent that resistance to monotherapy would quickly emerge. Combinations of HIV-1-RT inhibitors were found to be superior and with the advent of protease inhibitors, two and three drug combinations have been used routinely.

The rationale for combining antiretroviral drugs includes several potential benefits including simultaneously targeting several distinct target sites which can impede the develop of resistant strains and potentially exploit synergistic combinations with enhanced efficacy and decreased toxicity thus reducing the quantity of each drug which must be administered. Simply combining drugs, however, does not necessarily result in synergy. Several factors that can effect drug interactions include pharmacokinetic considerations, binding affinity and potential competition for a particular target site.

### Drug Interaction Analyses—

For the in vitro cell-cell fusion studies, the possibility of either enhanced (synergy) or reduced (antagonism) efficacy of CCR5 antibodies in combination with CCR5 antagonists was considered. Models and approaches for the assessment of in vitro drug interactions have been described and reviewed (M. C. Berenbaum J. Theor. Biol. 1985 114:413-431, Pharmacol. Rev. 1989 41:93-141; W. R. Greco et al. Pharmacol. Rev. 1995 47:331-385; M. N Pritchard and C. Shipman Jr. Antivir. Res. 1990 14:181-205; J. Suhnel Antivir. Res. 1990 13:23-39.) Synergy and antagonism are defined as departure from the hypothesis that there is no interaction between two drugs. The Lowe additivity (LA) and Bliss independence (BI) theories are the two primary candidates for reference models which follow two different additive drug interaction theories.

Drug interaction models based on the LA theory assume that a drug cannot interact with itself. The concentrations of the drugs in combination are compared to the concentrations of the drugs alone that produce the same effect (S. Loewe, Arzneim Forsch. 1953 3:285-290). The relationship is described by the equation: 1=d_{A}/D_{A}+d_{B}/D_{B}, where d_{A} and d_{B} are the concentrations of drugs A and B in combination that elicit a certain effect (e.g. 50% inhibition). D_{A} and D_{B} are the iso-effective concentrations (e.g. IC₅₀) for each drug alone. The concentration response surface approach described by Greco et al. (Cancer Res. 1990 50:5318-5327) was used to analyze the data. A seven-parameter non-linear model (i) was fit to all experimental data including percent inhibitions calculated from replicates for all concentrations of the two drugs alone and in combination from two 384-well plates.

$$1 = \frac{{D}_{A}}{{{IC}_{50 A} ( \frac{E}{{E}_{max} - E} )}^{1 / mA}} + \frac{{D}_{B}}{{{IC}_{50 B} ( \frac{E}{{E}_{max} - E} )}^{1 / mB}} + α \frac{{D}_{A} * {D}_{B}}{{IC}_{50 A} {{IC}_{50 b} ( \frac{E}{{E}_{max} - E} )}^{0.5 ( 1 / mA + 1 / mB )}} \qquad ( i )$$

where Eₘₐₓ is the maximal response in a drug free control; IC_{50A} and IC_{50B} are the median inhibitory concentrations of drugs A and B, respectively, that produce 50% of the Eₘₐₓ; m_{A} and m_{B} are the slopes of concentration response curves for the drugs A and B, respectively; D_{A} and D_{B} are the drug concentrations for drugs A and B, respectively, as inputs in the above equation; E is the measured response at the drug concentrations D_{A} and D_{B}, as the output; and a is the drug interaction parameter which describes the nature of the interaction. The above equation was fit to the complete data set from experiment with unweighted least squares nonlinear regression using SAS program (SAS User's Guide: Statistics. 1999, 8^{th} Edition, SAS Institute, Cay, N.C.). The estimates of all seven parameters and their associated asymptotic standard errors and 95% confidence intervals were generated to interpret the results. In addition, the R², correlation and covariance matrices, and residual plots were checked for goodness of fit for the model.

Synergy is indicated when the parameter a was positive and its 95% confidence interval does not include 0. Antagonism is indicated when a was negative and its 95% confidence interval does not include 0. Loewe additivity or no interaction is indicated when the 95% confidence interval of a includes 0. Furthermore, the predicted additivity of the drugs combined was calculated by using all estimated parameters of the Greco model, except a that is fixed at 0. The deviance between the predicted response surface and the predicted additive surface is interpreted as percent synergy if the deviation is positive (i.e., if the response surface is above the additive surface), or percent antagonism if the deviation is negative i.e., the response surface is under the additive surface). A three-dimensional graph and a contour plot were generated to examine the magnitude of synergism as well as to determine the range of drug concentrations that produce synergism.

For the drug interaction models based on the BI theory (C. I. Bliss Ann. Appl. Biol. 1939 26:585-615) the estimates of effect of the drugs combined based on the effect of the drugs alone are compared with the observed data from experiment. Its relationship is described by the equation: I_{comb}=I_{A}+I_{B}−I_{A}*I_{B}, where I_{comb} is the predicted percent inhibition of the drugs A and B in combination that have no interaction. I_{A} and I_{B} are the observed percent inhibition of each drug alone. A three-dimensional approach developed by M. N Prichard and C. Shipman Jr. (Antivir. Res. 1990 14:181-205) was used to access the drug interactions. Theoretical additive interactions were calculated from the dose response curves of the individual drugs based on the Bliss Independence equation. For each combination of the two drugs in each plate, the observed percent inhibitions were subtracted from the theoretical additive percent inhibition to reveal greater than expected activities. The resulting surface would appear as a horizontal plane at 0% inhibition above the predicted additive surface if the interactions were merely additive. Any peaks above this plane would be indicative of synergy. Similarly, any depression in the plane would indicate antagonism. The 95% confidence intervals around the experimental dose response surface were used to evaluate the data statistically.

The total sum of differences between the observed percent inhibitions and the upper bound of 95% confidence interval of predicted additive percentages is calculated as a statistically significant synergy volume ΣSYN. The total sum of differences between the observed percent inhibitions and the lower bound of 95% confidence interval of predicted additive percentages is calculated as a statistically significant antagonism volume ΣANT. In general, the drug interaction is considered weak when the interaction volume is less than 100%. The interaction is considered moderate when the interaction volume is between 100% and 200%. And, the interaction is considered strong when the interaction volume is more than 200%.

Mouse anti-human CCR5 mAbs ROAb13 and ROAb14 were tested in the CCR5-mediated cell-cell fusion (CCF) assay, along with two other CCR5 mAbs 2D7 and 45523. Six antagonists were also tested in the CCF assay for IC₅₀ determinations. (TABLE 2)

As shown in TABLE 2, both ROAb13 and ROAb14 showed strong inhibitory effects in the CCF assay, with an IC₅₀ of 14 nM and 1.3 nM respectively. Antibody 2D7 also showed potent antiviral activity (IC₅₀=4.3 nM), however, mAb 45523 exhibited relatively weak inhibitory effects on cell-cell fusion (IC₅₀=23 nM).

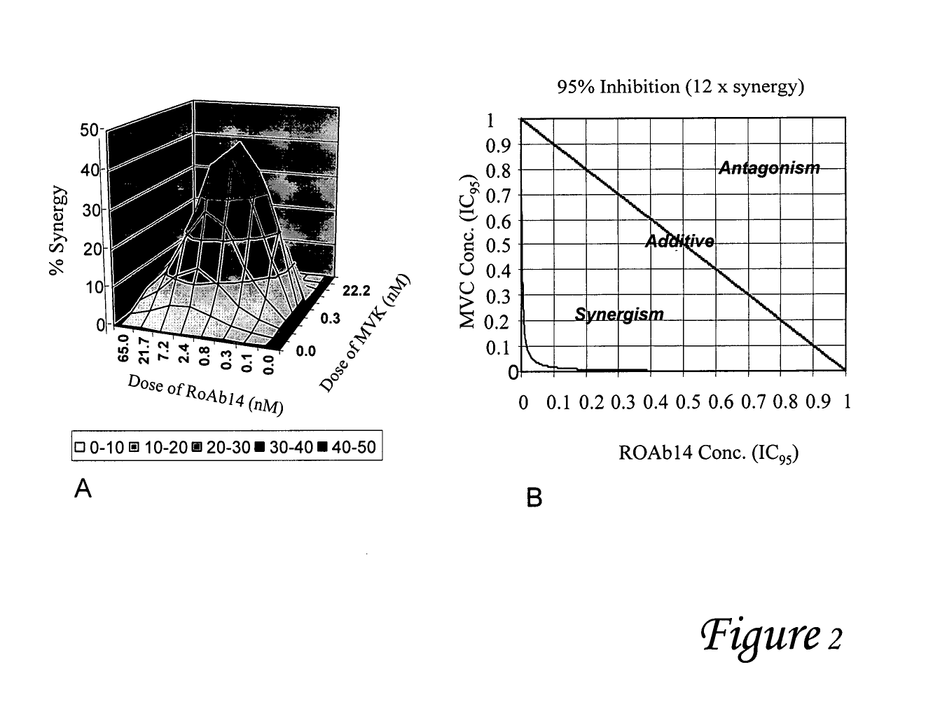

Seven-point half-log dilutions of CCR5 ROAb14 and ten-point half-log dilutions of CCR5 antagonist 4 were tested in CCF assay, alone or in various dose combinations. The inhibitory effects of each dose point were calculated and indicated as percent inhibition. Strong synergy is evident between ROAb14 and MVC on cell-cell fusion. For example, when MVC and ROAb14 were added alone both at 0.27 nM, 13% and 12% of inhibition was obtained, respectively. However, when these two drugs were added together at the same concentrations, 42% inhibition was observed, which is 19% higher than the predicted additive 23% inhibition based on the Bliss Independence equation. Furthermore, 16% synergy with 95% confidence was calculated under this dosing combination. Similarly, the percent synergy with 95% confidence was calculated for all checkerboard dosing points and a 3D graph was generated, which suggested a significant synergy at wide dose ranges for both drugs ROAb14 and MVC (FIG. 2). The interaction parameter a of the fully parametric Greco's model was positive (24.8±2.8), and the 95% confidence interval did not overlap 0, indicating a statistically significant synergy. When the interaction was determined based on Bliss Independence theory using the Prichard model, a strong synergy was also suggested (TABLE 3), with a 385% synergy volume (95% ΣSYN). No antagonistic effects were observed.

The data for RoAb14A and MVC is also plotted in FIG. 2 as an isobologram which provides a 2-dimensional graphical representation of the level of synergy at a specific level of inhibition. The isobologram is calculated from a seven-parameter non-linear model (ii) proposed by Greco et al. (Cancer Res. 1990 50:5318-5327) fits all experimental data, including % inhibitions calculated from replicates, for all concentrations of the two drugs alone and in combination from two 384-well plates. Then, the isobologram is calculated in the form:

$$\frac{{D}_{A}}{{D}_{x , A}} = \frac{1 - \frac{{D}_{B}}{{D}_{x , B}}}{1 + \frac{α * {D}_{B}}{{D}_{x , B}} {( \frac{100 - X}{X} )}^{0.5 ( 1 / mA + 1 / mB )}} \qquad ( ii )$$

where Dx,_{A} and Dx,_{B} are the estimated concentrations of drugs A and B, respectively, that produce X % inhibition (e.g., 10, 50, 90% inhibition); m_{A} and m_{B} are the slopes of concentration response curves for the drugs A and B, respectively; D_{A} and D_{B} are the drug concentrations for drugs A and B, respectively; and a is the drug interaction parameter. The isobologram is calculated and plotted using SAS program (SAS User's Guide: Statistics 1999, 8^{th} Edition, SAS Institute, Cay, N.C.). The equation of the isobologram is a hyperbola. The isobologram generated at the 95% inhibition level is depicted in FIG. 2. A diagonal straight line is expected if only additive effect is observed, and an inward curve toward the low doses indicates synergism and an outward curve indicates antagonism. The closer the curve toward the low doses, the higher the synergy is, and the smaller the doses of the drugs in combinations are needed to achieve that given inhibition.

Synergism allows lower doses of the antibody and antagonist to be used in combination than would be required based upon efficacy of each compound alone. For instance, to reach 95% inhibition, 65 nM and 22.2 nM of ROAb14 and MVC, respectively, were required; however, if both drugs were added together, only 0.8 nM of ROAb14 plus 2.47 nM of MVC were required to achieve 95% inhibition. A reduction of 81-fold in ROAb14 dose or 9.8-fold in MVC dose was observed in this case.

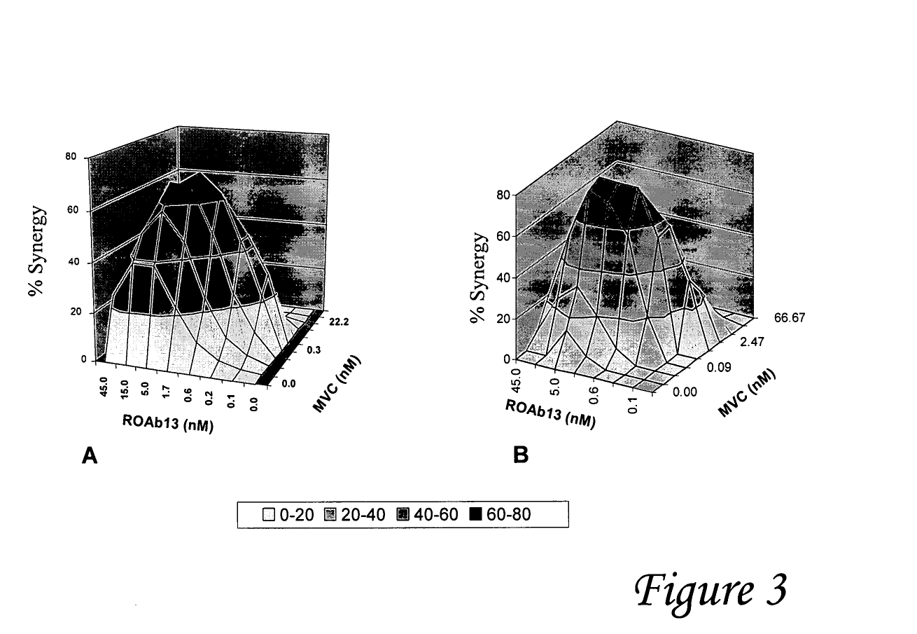

ROAb13, which binds to the N-terminal end of CCR5 exhibited approximately 60% higher synergy than ROAb14 when combined with the same CCR5 antagonist MVC (FIG. 3). The α parameter for the ROAb13-MVC combination was calculated using the Greco's model as 662±99 (TABLE 3), which is much higher than that for the ROAb14-MVC combination (24.8±2.8). Similarly, a 1,314% synergy volume (95% (ΣSYN) resulted from Prichard's model was much higher than that for the ROAb14-MVC combination (ΣSYN=385%). Furthermore, this synergistic effect occurs at very wide dose ranges for both ROAb13 and MVC, indicating a true potent synergy.

Other CCR5 antagonists including SCH-D, AK602, and novel antagonists 4, 5 and 6, were also tested for their interactions with various antibodies in the CCF assay system. These antagonists possess distinct structures but all exhibited potent antiviral activities. Both Greco's model and Prichard's model were used to analyze the drug interactions for these different combinations and the results were summarized in TABLE 3. Among all the CCR5 antagonists tested, AK602 exhibited the highest synergy when in combination with ROAb14 or ROAb13.

TABLE 3

| | |
|---|---|
| Greco Model | Prichard Model |

| Drug 1 | Drug 2 | α ± SE | ΣSYN | ΣANT |
|---|---|---|---|---|
| RoAb14 | AK602 | 126.9 ± 58.8 | 769 | −2 |
| | MVC | 24.8 ± 2.8 | 385 | −17 |
| | SCH-D | 20.6 ± 1.7 | 308 | −11 |
| | 4 | 20.7 ± 2.6 | 398 | −17 |
| | 5 | 16.7 ± 3.1 | 286 | −7 |
| | 6 | 9.8 ± 1.8 | 165 | −5 |
| | Median | 36.6 | 385.2 | −9.8 |
| RoAb13 | AK602 | 3296.3 ± 1113.2 | 1612 | 0 |
| | MVC | 662.3 ± 99.5 | 1314 | −1 |
| | SCH-D | 555.2 ± 87.0 | 1164 | −3 |
| | 4 | 183.6 ± 24.6 | 995 | −8 |
| | 5 | 2214.2 ± 568.9 | 2034 | −5 |
| | 6 | 215.3 ± 61.6 | 1144 | 0 |
| | Median | 1187.8 | 1377.2 | −3 |
| 2D7 | MVC | 13.2 ± 1.5 | 298 | −1 |
| | AK602 | 2.1 ± 0.6 | 113 | −1 |
| | 6 | 0.3 ± 0.2 | 45 | −36 |
| | Median | 5.2 | 152 | −16.7 |
| 45532 | MVC | −0.03 ± 0.008 | 3 | −102 |
| | AK602 | −0.03 ± 0.007 | 2 | −114 |
| | Median | −0.03 | 2.5 | −108 |

Strong synergy was not observed with all anti-CCR5 antibodies and the potent synergism between RoAb 13 and RoAb14 was unexpected. Murine CCR5 mAb 2D7, which is reported to bind to the N-terminal half of extracellular loop 2 (ECL2) of CCR5, exhibited weak to moderate synergy in combination with CCR5 antagonist MVC and AK602. The α parameters for 2D7-MVC and 2D7-AK602 combinations were determined to be 13.2 and 2.1, respectively by using Greco's model. These values were much smaller than that for the ROAb13-MVC or ROAb14-MVC combinations (TABLE 3). Another commercially available anti-CCR5 mAb 45523 that was previously shown to bind multiple exodomains of CCR5 was also investigated for its interactions with CCR5 antagonists in cell-cell fusion assay. As shown in TABLE 3, the α parameter and ΣSYN for 45523-MVC combination were −0.03 and 3, respectively, suggesting no synergism between 45523 and MVC. The CCR5 antagonist AK602 completely blocked the binding of mAb 45523 (K. Maeda et al. J. Virol. 2004 78:8654-62)

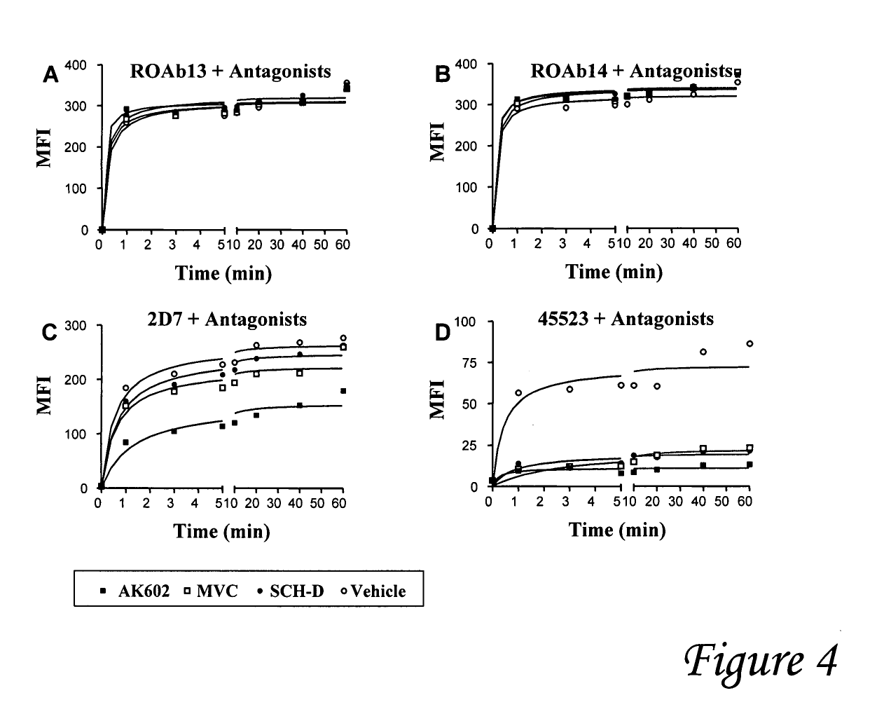

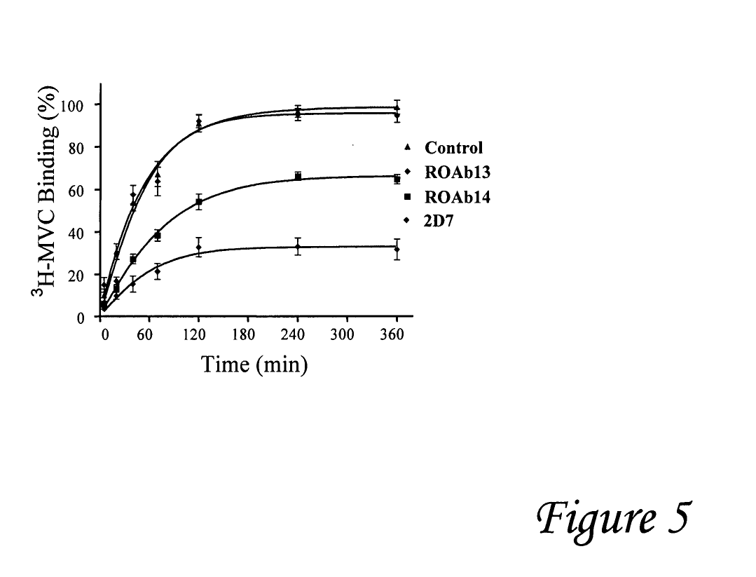

The potential for synergism should be maximized when both the antibody and the low molecular weight antagonist (or fusion or attachment inhibitor) can bind independently to the CCR5 receptor. The precise position of the epitope and the potential for allosterically induced conformational changes make predictions of independent binding hazardous. Surprisingly RoAb13 and RoAb14 binding were unaffected by pre-incubation with and the continued presence of CCR5 antagonists MVC, AK602, or SCH-D (FIGS. 4A and 4B). In contrast, the total binding of mAb 2D7 was partially inhibited by pre-incubation of CHO—CCR5 cells with antagonist AK602, MVC, and SCH-D (FIG. 4C). The total binding of 45523 was almost completely blocked by the three antagonists mentioned above, with its on-rate significantly reduced (FIG. 4D). Preincubation of CHO—CCR5 cells with mAbs and followed by competitive binding experiments demonstrated RoAb13 had no effect on MVC binding whereas RoAb14 and 2D7 exhibited 38 and 67% inhibition of binding (FIG. 5). Preincubation of the CCR5 receptor with AK602, MVC and SCH-D strongly inhibited 45523 binding by 75-85% (FIG. 4D) which is consistent with the failure to exhibit synergy.

Potent synergy also was observed between ENF and ROAb13 ( [symbol] =15.8±2.5), even greater synergy was observed between ENF and ROAb14 ( [symbol] =32.3±5.4) (TABLE 4). This result is in contrast to the mAb-antagonist interactions where much higher synergy was observed for ROAb13-antagonist combinations than ROAb14-antagonist combinations.

TABLE 4

| | |
|---|---|
| Greco Model | Pritchard Model |

| Drug 1 | Drug 2 | α ± SE | ΣSYN | ΣANT |
|---|---|---|---|---|
| ENF | RoAb14 | 32.0 ± 5.4 | 529 | −7 |
| ENF | RoAb13 | 15.8 ± 2.5 | 573 | −8 |
| ENF | 2D7 | 17.0 ± 5.0 | 246 | 0 |

### Pharmaceutical Formulations and Dosing

The present invention relates to a pharmaceutical composition comprising anti-CCR5 antibodies and low molecular weight allosteric CCR5 antagonists together with one or more pharmaceutical carriers. The components may be formulated separately in individual pharmaceutical compositions or in a unitary pharmaceutical composition containing both components. The present invention further relates to methods of treating or preventing HIV-1 using combination therapy with synergistic drug combinations. Combination therapy may be achieved by concurrent or sequential administration of the drugs. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent. The compounds may also be administered independently by different routes and each drug formulation may be individually optimized to provide optimal drug levels. Thus the antibody may be administered intravenously as a parental formulation and the low molecular weight compound may be administered as an orally in a solid or liquid formulation.

To prepare pharmaceutical compositions for use in accordance with the invention, an effective amount of a particular compound, in base or acid addition salt form, as the active ingredient is combined in intimate admixture with a pharmaceutically acceptable carrier, which carrier may take a wide variety of forms depending on the form of preparation desired for administration. As used herein “pharmaceutically acceptable carrier” includes any and all solvents, dispersion media, coatings, antibacterial or antifungal agents, isotonic and absorption delaying agents and the like that are physiologically compatible. These pharmaceutical compositions are desirably in unitary dosage form suitable, preferably, for administration orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent a convenient oral dosage unit form for the low molecular weight antagonist, in which case solid pharmaceutical carriers are obviously employed. The low molecular weight antagonist can also be combined with the antibody in a parenteral formulation. For parenteral compositions, the carrier will usually comprise sterile water, at least in large part, though other optional ingredients including pharmaceutically acceptable carriers, excipients or stabilizers, to aid solubility for example, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed.

Formulations for parenteral administration must be sterile solutions which can be achieved by filtration of the solution through sterile filtration membranes.

Acceptable carriers, excipients, or stabilizers are nontoxic to recipients at the dosages and concentrations employed, and include buffers such as acetate, TRIS, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g. Zn-protein complexes); and/or non-ionic surfactants such as TWEEN™, PLURONICS™ or polyethylene glycol (PEG). The antibody preferably comprises the antibody at a concentration of between 5-200 mg/mL.

Actual dosage levels of the active ingredients in the pharmaceutical composition or treatment regime of the present invention may be individually varied so as to obtain an amount of the each active ingredient which is effective to achieve the desired therapeutic response for a particular patient, composition, mode of administration without being toxic to the patient. The selected dose range will depend on a variety of pharmacokinetic factors including the activity of the particular compositions of the present invention employed, or the ester, salt or amide thereof, the route of administration, the time of administration, the reate of excretion of the particular compounds employed, the age, sex, weight, condition, general health and prior medical history of the patient undergoing treatment and other factors well known in the medical arts.

### EXAMPLE 1

### Preparation of Monoclonal Antibodies

Antibodies were prepared by giving female Balb/c mice a primary intraperitoneal immunization with 10⁷ CCR5-expressing cells (CHO—CCR5 or L1.2-CCR5) with complete Freund's adjuvant. The second immunization was done 4-6 weeks later similarly except incomplete Freund's adjuvant was used with the cells.

The mice were then boosted at 4-6 week intervals with 10⁷ CHO—CCR5 or L1.2-CCR5 cells with no adjuvant. The last immunization was carried out intraperitoneally with 10⁷ CCR5-expressing cells or intravenously with 2×10⁶ CCR5-expressing cells on the 3rd or 4th day before fusion. The spleen cells of the immunized mice were fused with myeloma cells according to Galfré (Galfre, G. and C. Milstein, Preparation of monoclonal antibodies: strategies and procedures in Methods Enzymol. 1981 73(Pt B):3-46.). Briefly, about 1×10⁸ spleen cells of the immunized mouse were mixed with the same number of myeloma cells P3×63-Ag8-653 (ATCC, Manassas, Va.), fused and cultivated in HAZ medium (RPMI 1640 containing 10% FCS, 100 mM hypoxanthine, and 1 μg/ml azaserine). Ten days after fusion, the supernatants were tested for specific antibody production. Hybridomas that produced the most potent supernatants in inhibiting CCR5-mediated cell-cell fusion were then cloned by limiting dilutions.

### EXAMPLE 2

### CCR5-Mediated CCF Assay

CCF assay was performed as described before (C. Ji, J. Zhang, N. Cammack and S. Sankuratri, J. Biomol. Screen. 2006 11(6):652-663). Hela-R5 cells (express gp160 from R5-tropic virus and HIV-1 Tat) were plated in 384 well white culture plates (BD Bioscience, Palo Alto, Calif.) at 7.5×10³ cells per well in phenol red-free Dulbecco's Modified Eagle Medium (DMEM) supplemented with 10% FBS, 1× Pen-Strep, 300 μg/mL G418, 100 μg/mL hygromycin, and 1 μg/mL doxycycline (Dox) (BD Bioscience, Palo Alto, Calif.), using Multimek (Beckman, Fullerton, Calif.) and incubated at 37° C. overnight to induce the expression of gp160. Ten μL diluted compounds in medium containing 5% DMSO were added to the cells, followed by the addition of CEM-NKr—CCR5-Luc (obtained from NIH AIDS Research & Reference Reagents Program) that expresses CD4 and CCR5 and carries a HIV-2 long terminal repeat (LTR)-driven luciferase reporter gene at 1.5×10⁴ cells/15 μL/well and incubated for 24 hrs. At the end of co-culture, 15 μL of Steady-Glo luciferase substrate was added into each well, and the cultures were sealed and gently shaken for 45 min. The luciferase activity were measured for 10 sec per well as luminescence by using 16-channel TopCount NXT (PerkinElmer, Shelton, Conn.) with 10 min dark adaptation and the readout is count per second (CPS). For the drug interaction experiments, small molecule compounds or antibodies were serially diluted in serum-free and phenol red-free RPMI containing 5% dimethyl sulfoxide (DMSO) (CalBiochem, La Jolla, Calif.) and 1×Pen-Strep. Five μL each of the two diluted compound or mAb to be tested for drug-drug interactions were added to the Hela-R5 cells right before the addition of target cells. The checker board drug combinations at various concentrations were carried out as shown in FIG. 1A.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A pharmaceutical composition for treating an HIV-1 infection, or preventing an HIV-1 infection, or treating AIDS or ARC, comprising co-administering to a patient in need thereof a therapeutically effective amount of a synergistic combination comprising an isolated antibody which antibody binds to the CCR5 receptor and wherein the CDR3 of the variable heavy chain sequence of said antibody is SEQ ID NO. 9 or 10, and a CCR5 antagonist, a viral fusion inhibitor or a viral attachment inhibitor.

2. A pharmaceutical composition according to claim 1 where said viral fusion inhibitor is enfuviritide said viral attachment inhibitor is TNX-355 or said CCR5 antagonist is selected from the group consisting of TAK-220, TAK-779, AK602(ONO 4128), SCH—C, SCH-D, MVC, Ia, Ib, Ic and Id. 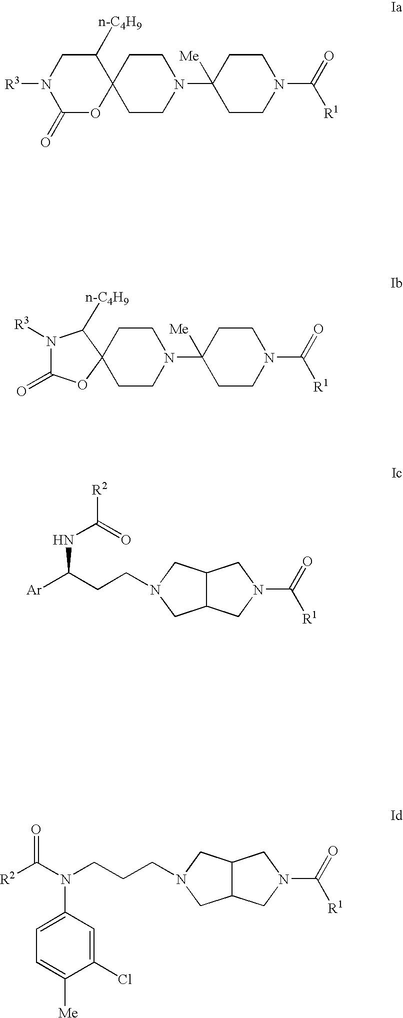
wherein
Ar is phenyl, 3-fluorophenyl, 3-chlorophenyl or 3,5-difluorophenyl;
R¹ is selected from the group consisting of: 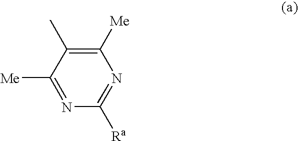
wherein R^{a} is hydrogen, —OH, —NMeCH₂CONH₂ or —OCMe₂CONH₂; 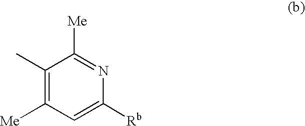
wherein R^{b} is hydrogen or cyano; 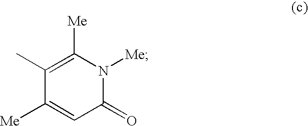
and, 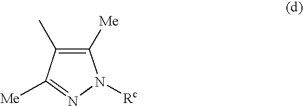
wherein R^{c} is 6-trifluoromethylpyridazin-3-yl, pyrimidin-5-yl, 5-trifluoromethyl-pyridin-2-yl;
R² is selected from the group consisting of cyclopentyl, 2-carboxy-cyclopentyl, 3-oxo-cyclopentyl, 3-oxo-cyclohexyl, 3-oxo-cyclobutyl, 3-oxa-cyclopentyl, 2-oxa-cyclopentyl, 4,4-difluorocyclohexyl, 3,3-difluoro-cyclobutyl, N-acetyl-azetidin-3-yl, N-methylsulfonyl-azetidin-3-yl and methoxycarbonyl;
R³ is selected from the group consisting of cyclohexyl methyl, tetrahydro-pyran-4-yl methyl; 4-methoxy-cyclohexanyl, 4-fluoro-benzyl, 4,4-difluorocyclohexyl-methyl, 2-morpholin-4-yl-ethyl and N—C₁₋₃ alkoxycarbonyl-piperidin-4-yl methyl; or,
pharmaceutically acceptable salts thereof.

3. A pharmaceutical composition according to claim 2 wherein said CCR5 antagonist is selected from the group consisting of I-1, I-2, I-3, I-4, I-5, I-6, I-7, I-8, I-9, I-10, I-11, I-12, I-13, I-14, I-15, I-16, I-17, I-18, I-19, I-20, I-21 and I-22.

4. A pharmaceutical composition according to claim 1 wherein said isolated antibody has a variable heavy chain sequence and a variable light chain sequence selected form the group consisting of:
(a) said variable heavy chain sequence is SEQ ID NO: 1 and said variable light chain sequence is SEQ ID NO: 2;
(b) said variable heavy chain sequence is SEQ ID NO: 3 and said variable light chain sequence is SEQ ID NO: 4;
(a) said variable heavy chain sequence is SEQ ID NO: 5 and said variable light chain sequence is SEQ ID NO: 6; and,
(a) said variable heavy chain sequence is SEQ ID NO: 7 and said variable light chain sequence is SEQ ID NO: 8.

5. A pharmaceutical composition according to claim 4 wherein said CCR5 antagonist is selected from the group consisting of TAK-220, TAK-779, AK602(ONO 4128), SCH—C, SCH-D, Ia, Ib, Ic and Id wherein Ar, R¹, R² and R³ are as defined previously.

6. A pharmaceutical composition according to claim 4 wherein said viral fusion inhibitor is enfuviritide.

7. A pharmaceutical composition according to claim 4 wherein said viral attachment inhibitor is TNX-355.

8. A composition according to claim 1 wherein said antibody is produced by a hybridoma cell line selected from the group consisting of m<CCR5>Pz01.F3, m<CCR5>Px04.F6, m<CCR5>Pz03.1C5 and m<CCR5>Px02.1C11.

9. A method for treating an HIV-1 infection, or preventing an HIV-1 infection, or treating AIDS or ARC, comprising co-administering to a host in need thereof a therapeutically effective amount of a synergistic combination of an isolated antibody which antibody binds to the CCR5 receptor and wherein the CDR3 of the variable heavy chain amino acid sequence of said antibody is selected from the group consisting of SEQ ID NO. 9 or 10, and a CCR5 antagonist, a viral fusion inhibitor or a viral attachment inhibitor.

10. A method according to claim 9 wherein said CCR5 antagonist is selected from the group consisting of TAK-220, TAK-779, AK602(ONO 4128), SCH—C, SCH-D, Ia, Ib, Ic and Id wherein Ar, R¹, R² and R³ are as defined previously.

11. A method according to claim 9 wherein said viral fusion inhibitor is enfuviritide.

12. A method according to claim 9 wherein said viral attachment inhibitor is TNX-355.

13. A method according to claim 9 wherein said isolated antibody has a variable heavy chain sequence and a variable light chain sequence selected form the group consisting of:
(a) said variable heavy chain sequence is SEQ ID NO: 1 and said variable light chain sequence is SEQ ID NO: 2;
(b) said variable heavy chain sequence is SEQ ID NO: 3 and said variable light chain sequence is SEQ ID NO: 4;
(a) said variable heavy chain sequence is SEQ ID NO: 5 and said variable light chain sequence is SEQ ID NO: 6; and,
(a) said variable heavy chain sequence is SEQ ID NO: 7 and said variable light chain sequence is SEQ ID NO: 8.

14. A method according to claim 13 wherein said antibody is produced by a hybridoma cell line selected from the group consisting of m<CCR5>Pz01.F3, m<CCR5>Px04.F6, m<CCR5>Pz03.1C5 and m<CCR5>Px02.1C11.

15. A method according to claim 14 wherein said CCR5 antagonist is selected from the group consisting of TAK-220, TAK-779, AK602(ONO 4128), SCH—C, SCH-D, MVC Ia, Ib, Ic and Id wherein Ar, R¹, R² and R³ are as defined previously.

16. A method according to claim 14 wherein said viral fusion inhibitor is enfuviritide.

17. A method according to claim 14 wherein said viral attachment inhibitor is TNX-355.
